(19) United States
(12) Patent Application Publication
(10) Pub. No.: US 2020/0013501 A1
(43) Pub. Date: Jan. 9, 2020

(54) PREDICTIVE MEDICAL EQUIPMENT MAINTENANCE MANAGEMENT

(71) Applicant: General Electric Company, Schenectady, NY (US)

(72) Inventors: John Page, Verona, WI (US); Brandon Henak, Wauwatosa, WI (US); Kevin Tissot, Brooklyn, WI (US); Guy Vesto, Kildeer, IL (US)

(73) Assignee: General Electric Company, Schenectady, NY (US)

(21) Appl. No.: 16/030,247

(22) Filed: Jul. 9, 2018

(51) Int. Cl. G16H 40/40; G08B 21/24

(52) U.S. Cl. CPC G16H 40/40; G08B 21/24

(57) ABSTRACT

Systems and methods of managing maintenance for a plurality of monitored medical devices include receiving streaming time series medical device data from the plurality of monitored medical devices. The streaming time series medical device data is analyzed to determine an operational status of a component of a medical device of the plurality of monitored medical devices. A maintenance procedure for the medical device is determined from the operational status of the component of the medical device.

### BACKGROUND

The present disclosure is related to the field of data processing and analytics. More specifically, the present disclosure is related to predictive scheduling based upon collected and processing of medical device data.

Medical devices generate large amounts of data from sensors and inputs that are used in real-time on the device to display relevant information or to provide alerts to users of the device. In some settings, this data is made available to external systems through proprietary or standards-based data messaging. Examples of these systems include hospital information systems (HIS) and/or electronic health records (EHR). HIS and EHR systems collect and process a variety of information, including patient data. HIS systems seek to coordinate the administrative, financial, legal needs of a hospital system, which may include the management of patient data or particular departments within a hospital system or network, for example laboratory services and patient imaging. EHR systems seek to collect and coordinate health information generated both on a patient by patient basis as well as population information. In either case of HIS or EHR systems, these currently available systems only connect to medical devices in a limited manner and to the extent that information directly from medical devices is incorporated into these systems, such information is typically the physiological data directly acquired from such medical devices, e.g. heart rate, pulse rate, blood oxygenation, blood pressure, temperature, respiration rate, etc.

However, medical devices as typically used in hospitals produce significantly more data than merely the monitor physiological parameters of a patient. Therefore, new systems and methods are desirable to collect and process this medical device data and to incorporate information gained from these systems into hospital system workflows and decision making.

### BRIEF DISCLOSURE

An exemplary embodiment of a method of managing a plurality of monitored medical devices includes receiving streaming time series medical device data from the plurality of monitored medical devices. The streaming time series medical device data is analyzed to determine an operational status of a component of a medical device of the plurality of monitored medical devices. A maintenance procedure for the medical device is determined from the operational status of the component of the medical device. A notification is produced of the determined maintenance procedure.

An exemplary embodiment of a system for processing streaming time series of medical device data. The system includes a data ingestion module that receives streaming time series medical device data and preprocesses the streaming time series medical device data. A medical device data processor receives the streaming time series medical device data and applies a plurality of streaming analytics rules to the streaming time series medical device data to identify an operational status of a component of a medical device in the streaming time series medical device data. The medical device data processor determines a maintenance procedure for the medical device from the operational status of the component of the medical device. A maintenance scheduler incorporates a schedule of use of the monitored medical devices with a scheduled time for the determined maintenance procedure.

### BRIEF DESCRIPTION OF THE DRAWINGS

FIG. 1 is a system diagram of an exemplary embodiment of a system for medical device data collection and processing.

FIG. 2 depicts an exemplary embodiment of a method of medical device data processing.

FIG. 3 depicts an exemplary embodiment of a method of medical device data curation and learning.

FIG. 4 depicts an exemplary embodiment of a fleet management dashboard.

FIG. 5 depicts an exemplary embodiment of a monitored room status dashboard.

FIG. 6 is a system diagram of an exemplary embodiment of a system for medical device data collection and processing.

FIG. 7 depicts an exemplary embodiment of a fleet management dashboard.

FIG. 8 is a flow chart that depicts an exemplary embodiment of a method of managing maintenance of medical devices.

### DETAILED DISCLOSURE

Embodiments of systems and methods as disclosed herein operate to collect and process a wide variety of medical device data. Medical device data includes physiological data that is acquired from a patient by a medical device and machine data collected internally from the medical device itself. Machine data can include alarms, device status, settings, messages, and measured operational data. Machine data may further include settings and values that represent specific actions taken with the medical device for example, in response to automated controls or due to clinician inputs. For example, in an anesthesia delivery machine, this may include changes to oxygen and/or anesthetic agent concentrations. The machine data may further include clinical and/or technical alarms initiated by the medical device or device diagnostic information. Still further examples of the machine data include proactive or predictive service alerts from the medical device, maintenance checkout information and/or processor clock cycle signals or power signals or other operational signals from various components of the medical device indicative that the medical device is turned on, in use, in operation, held in a stand by condition, or turned off.

This machine data can be collected in time series format as provided from the medical devices themselves. As used herein, the time series format of the medical device data can include waveforms, binary data, numeric data, and/or textual data in a time series format. Embodiments of the systems and methods as disclosed herein receive the medical device data from the medical devices at a frequency similar to the frequency at which it is produced by the medical device. In embodiments, this increased velocity of the received data and the monitoring and analysis of medical device machine data can enable improved monitoring systems and method as disclosed herein. As described in further detail herein embodiments of systems and methods support high speed data ingestion, enrichment, normalization, and data curation of the medical device data. The medical device data can undergo real time analysis and further enrichment of the data with event detection and notation. While all of the medical device data can be saved for retrospective and automated machine learning and analysis, event detection and notation can be used to create further exemplary files of medical device data stemming from particular events or conditions which can be used as exemplary or case study data for further analysis.

In embodiments, machine learning and data analysis of the collected medical device data can be used to develop and refine control algorithms, early detection/predictive algorithms, and/or alarm algorithms to improve clinician use of the medical devices, hospital workflow, operation or alarm settings of the medical devices themselves, or event detection within the system.

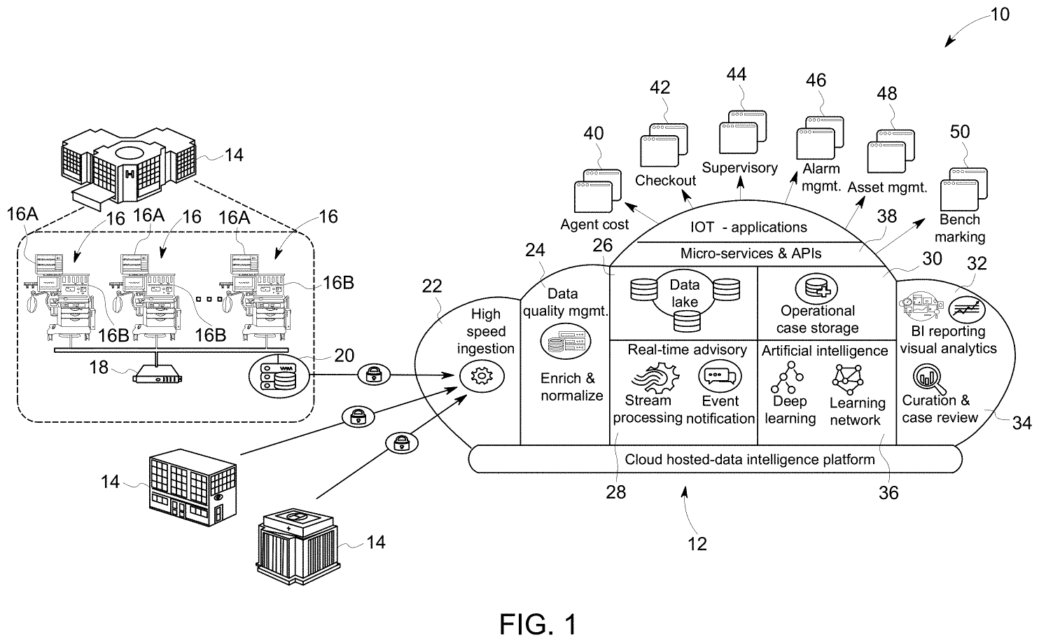

FIG. 1 depicts an exemplary embodiment of a system 10 for medical device data collection and processing. The system 10 includes a medical device data (MDD) processing system 12. The MDD processing system 12 can be implemented in a variety of hardware and/or software implementations, it should be noted that such implementations are not considered to be limiting. For example, it is contemplated that any or all of the MDD processing system 12 may be embodied exclusively in hardware, exclusively in software, exclusively in firmware or in any combination of hardware, software, and/or firmware. While the following describes exemplary methods and systems, the examples provided herein are not the only way to implement such methods and systems.

In embodiments wherein any of the claims are read to cover an entirely software and/or firmware implementation, in any embodiment, at least one of the elements is hereby expressly defined to include a tangible and non-transient computer readable medium. As used herein, the term tangible computer readable medium is expressly defined to include any type of computer readable storage and to exclude propagating signals. Additionally or alternatively, the example methods and systems may be implemented using coded instruction (e.g., computer readable instructions) stored on a non-transitory computer readable medium such as a flash memory, a read-only memory (ROM) a random-access memory (RAM), a cache, or any other storage media in which information is stored for any duration (e.g. for extended period time periods, permanently, brief instances, for temporarily buffering, and/or for caching of the information). As used herein, the term non-transitory computer readable medium is expressly defined to include any type of computer readable medium and to exclude propagating signals.

In exemplary and non-limiting embodiments of the medical device data processing system 12, the system 12 is implemented by one or more networked processors or computing devices. Processing system 12 may be implemented in a cloud computing platform and/or infrastructure. Memory and processors as referred to herein can be standalone or integrally constructed as part of various programmable devices, including for example, computers or servers. Computer memory of computer readable storage mediums as referenced herein may include volatile and non-volatile or removable and non-removable media for a storage of electronic-formatted information such as computer readable program instructions or modules of computer readable program instructions, data, etc. that may be stand-alone or as part of a computing device. Examples of computer memory may include, but are not limited to RAM, ROM, EEPROM, flash memory, CD-ROM, DVD-ROM or other optical storage, magnetic cassettes, magnetic tape, magnetic disc, or other magnetic storage devices, or any other medium which can be used to store the desired electronic format of information and which can be accessed by the processor or processors or at least a portion of a computing device.

The MDD processing system 12 is communicatively connected to at least one hospital network 14. Such communicative connections as well as the hospital network itself may include, but are not limited to, a wide area network (WAN); a local area network (LAN); the internet; wired or wireless (e.g. optical, Bluetooth, radio frequency (RF) network; a cloud-based computer infrastructure of computers, routers, servers, gateways, etc.; or any combination thereof associated therewith that allows the system or portions thereof to communicate with one or more computing devices.

The hospital network 14 may exemplarily be a network associated with a portion of a hospital, for example a surgery unit or department of a hospital, or may be more broadly located across medical devices of an entire hospital. It further will be recognized that while some embodiments and implementations of the systems and methods as disclosed herein may seek to operate on a single hospital or unit of a hospital, still other embodiments may connect a plurality of hospital networks, including hospitals currently owned or operated or otherwise affiliated with one another. In still further embodiments while individual hospitals or groups of hospitals may use the MDD processing system 12, the MDD processing system 12 receives and processes information from a plurality of hospital networks including those unaffiliated with one another at the same time.

As depicted in FIG. 1, the hospital network 14 includes a plurality of medical devices 16. The medical devices 16 exemplarily include physiological monitoring devices 16a as well as patient therapy devices 16b. Physiological monitoring devices 16a may include, but are not limited to heart rate monitors, blood pressure oxygenation monitors, respiration monitors, ECG monitors, EEG monitors, or EMG monitors. An exemplary embodiment of an anesthesia delivery machine will be used for discussion purposes as the medical device, and more specifically as the patient therapy device 16b, although it will be recognized by a person of ordinary skill in the art that other devices, including, but not limited to patient respiratory assistance devices or dialysis machines may be further non-limiting examples of patient therapy devices. However, it will be recognized that therapy devices may also include capabilities to not only deliver patient therapy, but also to measure physiological parameters of a patient. For example, embodiments of anesthesia delivery machines may include gas analysis modules operable to measure gas concentrations expired by the patient. In other exemplary embodiments, imaging devices, including, but not limited to X-ray, CT, MRI, and ultrasound devices may be examples of medical devices 16 as contemplated within the present disclosure. Still further examples of medical devices may include video and/or audio recording devices.

In an exemplary embodiment, a limited version of the MDD processing system 12 as described herein may be implemented locally, exemplarily as an anesthesia delivery management system 18. In such an embodiment, the anesthesia delivery management system 18 may operate to collect medical device data from a plurality of anesthesia delivery machines 16b for example, among other things as described herein, to monitor anesthesia agent use between anesthesia delivery machines and across procedures performed by those machines in an effort to visualize anesthetic agent consumption and use as well as to quantify, monitor, and evaluate trends across all of the anesthesia delivery machines in the hospital or surgical unit. In still further embodiments as disclosed herein, the medical device data can be used to manage the schedule and/or maintenance of medical devices used in the hospital or surgical unit.

The medical devices 16 are communicatively connected to one or more gateway devices 20. Gateway devices 20 may exemplarily be edge processing devices, cloud processing devices or internet gateways. The gateway 20 may exemplarily be an internet of things (TOT) gateway which facilitates a secure communications link between the medical devices 16 at the hospital network 14 with the servers, processors, and computer readable media implementing the MDD processing system 12. In exemplary embodiments, the gateway 20 may communicate directly with one or more of the medical devices 16, or may communicate with the medical devices 16 through an intermediate network, for example, the anesthesia delivery management system 18 or another medical device data system or network.

The gateway 20 receives the medical device data as time series data for any of the medical device data available from the medical devices. Exemplary embodiments as described herein will focus on machine data, except where specifically noted, although it will be recognized that other forms of medical device data may be used in similar manners as disclosed with respect to these exemplary embodiments. As noted above, the data streams of machine data are available in time series format as acquired from the medical devices and may include, but are not limited to time series information of alarms, device status, device settings, messages, and measured data. In embodiments, the medical devices may be equipped with sensors that improve the self-awareness of the medical device, e.g. sensors that monitor the function, inputs and/or outputs of various components of the medical device itself. Many such sensors are already incorporated into medical devices such as to measure compressor speeds and/or cycle times, internal pressures, voltages, clock speeds, or temperatures, or other sensors as will be recognized by a person of ordinary skill in the art or as disclosed in further detail herein.

The gateway 20 encrypts the time series formatted data and the encrypted data is transmitted using known wired and/or wireless communication techniques for encrypted data to the server, processors, and data storage carrying out the medical device data processing system 12. The gateway 20 continuously transmits de-identified medical device machine data in time series format over an encrypted communication channel to a high speed data ingestion module 22 of the MDD processing system 12. While the exemplary embodiment described herein may reference de-identified data, it will be recognized that other embodiments may use patient-identified data with appropriate considerations taken for handling patient data. The high speed data ingestion module 22 takes in the real time streams of medical device data. The data ingestion can be performed automatedly and preprocess the received streams of real time data in the time series for later processing by the MDD processing system 12. The high speed ingestion module 22 can receive concurrent data streams from multiple connected devices across multiple sites at a high incoming velocity, for example at or near the frequency at which medical devices can output machine data. In exemplary embodiments the high speed ingestion module 22 is scalable to continue to ingest increased bandwidth of medical device data without significant decrease in ingestion speeds.

The high speed ingestion module 22 takes the time series medical device data from the medical devices of one or more hospital networks and formats it for further processing by a data quality management module 24. In exemplary and non-limiting embodiments, the high speed injection module 22 supports open standard such as ASTMF 2761 or integrated clinical environmental (ICE). The data quality management module 24 exemplarily normalizes, enriches, and tags the data streams without negatively impacting data latency. In a healthcare environment, a variety of healthcare information products and/or systems may be used to provide medical services, collect medical data, conduct medical exams, etc. Such products and/or systems include the Centricity® suite of products and/or systems as offered by GE Healthcare®. However, many healthcare information systems operate using various messaging standards (e.g., Health Level 7 International (HL7 V2.x/v3), Clinical Document Architecture/Continuity Of Care Document (CDA/CCD), American Society for Testing Materials (ASTM), Digital Imaging and Communications in Medicine (DICOM), etc.)) and various standards and/or protocols (e.g., cross-enterprise document sharing (XDS.A/B) cross-enterprise document media interchange (XDM) cross-enterprise document reliable interchange (XDR), patient identifier cross-referencing/patient demographics query (PIX/PDQ) patient administration management (PAM), query for existing data (QED), national counsel for prescription drug programs (NCPDP), etc.)) that make system integration and/or communication more difficult. Thus, normalization may include reformatting of medical data to a consistent or compatible format for use within the MDD processing system 12. In an exemplary embodiment, the medical device data may be normalized into the ISO/IEEE 11073-10101 nomenclature and its extensions. In a still further exemplary embodiment, the data quality management module 24 can normalize the streams of incoming time series data by converting units of measure. The data quality management module 24 can further operate to identify and tag various types of medical device data, locations from which the medical device data was received, or time series data streams originating from the same medical device. These tags can be used as further detailed herein to identify and analyze groups of streams of time series data.

In an exemplary embodiment, the data quality management module 24 normalizes the received incoming data by transforming and/or translating the clinical data streamed from the source healthcare system or device into a canonical data model with associated metadata. The processed medical device data is stored in a data lake 26 which is exemplarily implemented in computer readable storage embodying capability to store terabytes of data. The data lake 26 is a long-term computer storage repository that holds large amounts of raw data in the it's native format until the data is needed. The native format may include the time series data from the medical devices which may be in waveform or binary format, audio data, image data, and/or video data. In embodiments, this can help to facilitate the ingestion of the data that while may not be processed in real time, may still be taken in in real time or near real time and instead stored in the data lake until further needed. This may be facilitated by identifying particular data streams and limiting the processing of those data streams, for example by the data quality management module 24, if it is known at that time that such data stream is not being used in real time analysis. In an exemplary embodiment, the data quality management module 24 may not convert the data to a canonical data model but may still attempt to tag, enrich, or index the data to facilitate later retrieval of that data in a standardized way from the data lake 26.

In a still further embodiment, portions of the data that are stored in the data lake 26 may also be additionally stored in a graph database which may be a separate database residing on the same computer readable storage, or may be embodied on separate computer readable storage from the data lake 26. The graph database may receive the data streams of which it is known that the system may analyze trends in that data stream. The graph database may store the streams of data in a time series format in a way that facilitates trending of the data over time and appending the data with events either identified in the data itself, in one or more of the other data streams, or received by the system from an external source. These events may include, but are not limited to medical device or clinician actions, clinical events, situations, or complications that arise during the medical procedure. The graph database may later be used by a clinician or technician to identify further relationships between trends and the data streams with other analysis as disclosed herein.

At the same time that the data is stored in the data lake 26, the enriched and normalized medical device data is provided to a stream processing engine 28 that uses artificial intelligence capability as described in further detail herein. The stream processing engine 28 identifies cases and events in the time series streams of medical device data. Identified clinical cases can be stored in an operational case database 30. Clinical cases may exemplarily include surgical and intensive care unit (ICU) cases. The clinical cases can be exemplarily identified by the medical device used and the timing of the medical data in the time series of the medical device data. For example, a time series of medical device data from an anesthesia delivery machine showing a change in status turning the machine and followed by changes to device settings and delivery and/or consumption of anesthetic agent all indicate that a clinical case has begun or is ongoing.

As noted above, the streams of time series medical device data originating from the same medical device or from the same location in a hospital may be tagged or otherwise identified as being related. These tags can be used to simultaneously analyze related data streams or combine analysis of related data streams to identify clinical cases. For example, a device status data stream analysis may be combined with a user input data stream, device setting data stream, and operational data streams to identify when the device is used and how it is used in the clinical case. This information may help to distinguish between a maintenance or checkout of the medical device by a technician from the use of the device for clinical case.

The analysis of the data streams of multiple medical devices, particularly those identified as being related or co-located can further be used to identify clinical cases. For example, coordinated or similar actions in data streams of an anesthesia delivery device and a related patient monitoring device, and/or respiratory support device and/or imaging device, etc. can further be used to identify that these devices are being used together for a clinical case. In still further embodiments, the streaming time series medical device data may be combined with information regarding scheduled clinical cases to help to further identify when and how the medical devices are used during clinical cases.

In embodiments, knowledge of a scheduled use of the medical device (e.g. anesthesia delivery machine) can be used to further identify clinical cases in the streams of medical device data. For example, input or received knowledge regarding a type and time of a scheduled procedure may help to identify the start and end of the clinical case in particular streams of medical device machine data. In an embodiment, a known schedule of use for the medical device can help to identify clinical cases from maintenance or calibration actions which may similarly require powering up and at least partial operation of the medical device.

The medical device data associated with the actions by the anesthesia delivery device and/or other medical devices during the identified clinical case are stored in the operational case database 30. In an example, the identification of the clinical case is stored along with the other time series streams of medical device data from that anesthesia delivery machine as well as time series streams of medical device data from any physiological monitors and/or other medical devices associated with the use of that anesthesia delivery machine. In another exemplary embodiment as described in further detail, a clinical case summary with links or identifiers to the associated time series medical device data stored in the data lake 26 can be created and stored in the operational case database 30.

In an embodiment, prior to storing the clinical cases in the clinical case database 30, the clinical cases may be classified or profiled which is a technique used for data curation. The profiling of the clinical cases may be based upon in part, the information in the clinical case summary, and as described in further detail herein, may be used to group the clinical cases into groups, for example normal cases, edge cases, and outlier cases. These determinations may be made in view of a comparison between the time series data in the clinical case against normal distributions of the same type of time series machine data in other similar clinical cases. Edge cases may be identified as borderline or ambiguous cases, not clearly defined as either normal or an outlier. In a merely exemplary embodiment, for a particular measured value or occurrence, a distribution of such occurrences may be used to establish normal, edge, and outlier cases. In a merely exemplary embodiment, a normal case may be within a standard deviation of a median value in the normal distribution while edge cases are between one and two standard deviations and outlier cases are greater than two standard deviations from the median. The categorized cases, as explained in further detail herein, for example, identified edge cases may be further investigated to create or improve event detection algorithms, rules for clinical decision support, alert algorithms, and predictive algorithms.

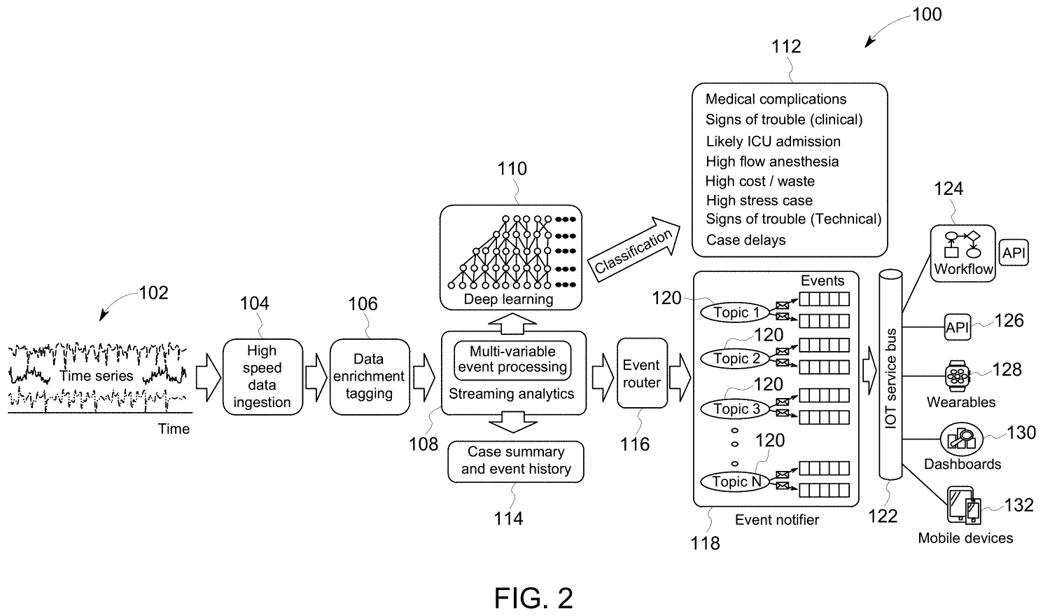

The stream processing engine 28 also identifies events in the time series streams of medical device data, for example in the manners as described in further detail herein and presented in business intelligence and visual analytics tools 32 which exemplarily may be presented on a graphical display communicatively connected to the medical device data processing system 12. The processing and reporting of events will be described in further detail herein with respect to FIG. 2.

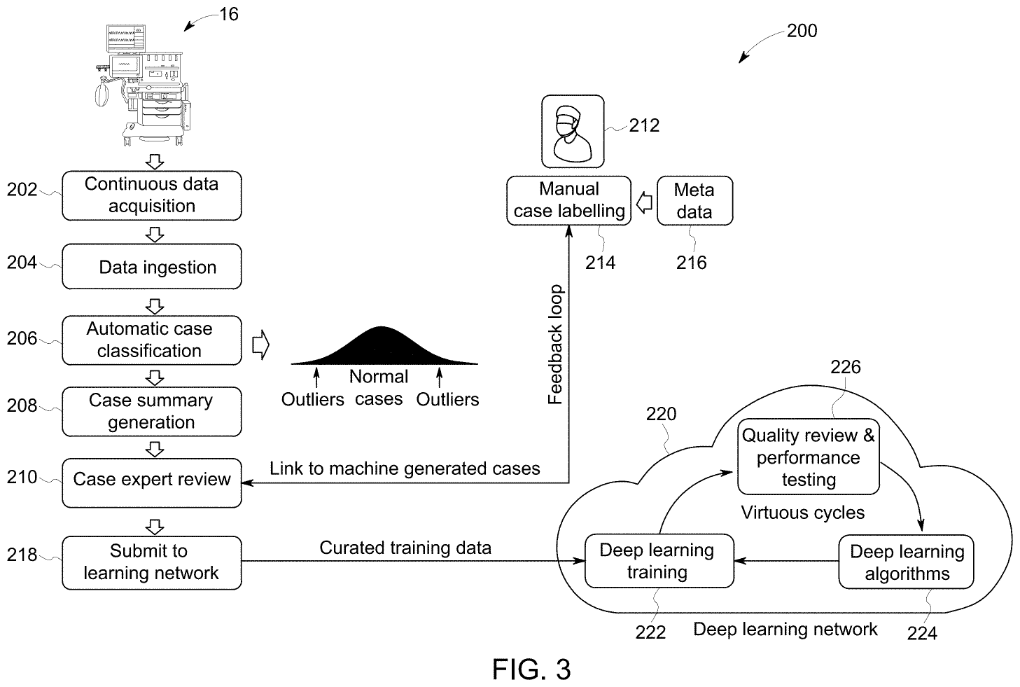

Once clinical cases are stored in the operational case database 30, clinical cases can be reviewed manually by a clinician or technician using a curation and case review tool. The curation and case review tool 34 can be presented in a graphical user interface on a graphical display and further provide inputs exemplarily through the graphical user interface for the user or technician to curate or otherwise assess the clinical cases. This can be performed for investigative, educational, and data curation purposes. Exemplary and non-limiting embodiments of the data curation will be described in further detail herein with respect to FIG. 3.

The reporting and visual analytics tool 32 can present the detected events in a variety of channels of communication. Exemplarily as will be explained in further detail herein, and including, but not limited to, the detected events can be presented visually through graphical user interfaces and graphical displays. The detected events or notifications of the detected events can also be reported by communication of events/event notifications to wearable or mobile devices and presentation of medical device data and identified events in visual form in reports and/or dashboards presented in a graphical user interface on a graphical display.

The results of the streaming analytics and event detection in the time series of medical device data can be provided to an application programming interface (API) 38 for use by application developers to provide monitoring, reporting, and/or control applications based upon the analyzed streams of medical device data. Such applications may operate through a computer operating system, a website browser, or operate on a mobile computing device or wearable computing device. Non-limiting examples of applications that may leverage the analysis of the time series medical device data include, but are not limited to an anesthetic agent cost dashboard 40, a checkout dashboard 42, a supervisory application 44, an alarm management application 46, an asset management application 48, and a benchmarking application 50.

The agent cost dashboard 40 exemplarily presents medical device data regarding anesthetic agent use across clinical cases as well as between anesthesia delivery machines within a hospital network or comparatively between hospital networks. By comparatively presenting this information anesthetic agent use and behavioral changes can be understood and undertaken to promote efficient use of anesthetic agent.

The checkout dashboard 42 may exemplarily assist in monitoring the inspection and maintenance of the monitored medical devices. Medical device data such as device status and settings, as well as messages and information in machine data may provide insight into the inspection processes for maintaining medical devices at a hospital network. The checkout dashboard may identify maintenance and/or testing events in the streams of machine data and note these identified testing events against a testing schedule, requirement (e.g. daily), or other criterion.

A supervisory application 44 may be used by attending and/or supervising anesthesiologists to more efficiently manage remote personnel and/or nurse anesthetists simultaneously working across multiple locations or theatres. An alarm management 46 application can report and present medical device data regarding alarm notifications and silences of alarm notifications in order to better understand and adjust sheen alarms to improve signal to noise in alarm events and to reduce alarm fatigue by clinicians.

Asset management applications 48 may present use, status, maintenance, and/or inspection information regarding medical devices (e.g. anesthesia delivery machines) or consumables used by medical devices, including components which while not replaced at every use must be frequently replaced, refilled, or refurbished during normal operation of the medical device (e.g. filters, absorbers). Benchmarking application 50 may provide further operational and quality performance across providers and/or organizations or in a comparative manner for example between hospital networks versus averages or between specific locations. Particular events and the application will be described in further detail herein.

FIG. 2 is a flow chart that depicts an exemplary embodiment of a method 100 of processing medical device data. An exemplary embodiment of the method 100 may be carried out by the medical device data processing system 12 as described above with respect to FIG. 1. As previously noted, embodiments of the systems and methods as described herein process medical device data as time series data of changes in each medical device data value 102. The data streams are acquired from the medical devices and may include time series format data regarding machine data such as alarms, device status, settings and values representing specific actions, messages, and/or measured data. The medical device data may further include patient physiological data obtained by sensors of the medical device. At 104 the time series data streams, multiple streams from each medical device in a medical device network are ingested to prepare the incoming data for processing and/or saving. In one exemplary embodiment, the medical device data is machine data. At 106 the processed medical device data is enriched by tagging the streams of data with identifying information and/or converting units of incoming medical device data to normalize units. The data enrichment and tagging at 106 is performed without negatively impacting latency of the ingested streams of medical device data. Streaming analytics are undertaken at 108 with a clustered streaming analytics engine capable of complex event correlation, data smoothing, and windowing operations. The streaming analytics is exemplarily performed by predictive algorithms from deep learning as will be described herein and in further detail with respect to FIG. 3.

In streaming analytics, data may be windowed into short segments against which the pattern recognition or predictive algorithms are applied. Further analysis can be applied between windowed portions of the time series and the windows overlap so that data is not missed and each segment of time series data is given both forward and backward context. As noted above, the analysis of the streaming time series data may not be limited to analysis of individual streams of data. Rather, different streaming analytics algorithms may relate the analysis of multiple streams of data, for example streams of data originating from the same medical device and/or streams of data originating from different medical devices but from the same location. This context may be provided by the tags added in the previous data enhancement. In still further embodiments, streaming analytics algorithms may analyze the streams of data in combination with other information as may be acquired from sources outside of the medical devices themselves. This other information may include but is not limited to time, date, hospital scheduling information or maintenance schedules in order to further carry out the clinical case identification and event detection as described herein.

The streaming analytic algorithms may exemplarily be alarm algorithms, control algorithms, event detection algorithms, or predictive algorithms. These algorithms may be predefined, or in other embodiments may be created as a result of automated learning, for example in the manners as described in further detail herein with respect to FIG. 3. Non-limiting examples of the streaming analytics algorithms may include early detection of deterioration of a patient's health status, detection of complications in an operating room (OR) or ICU, prediction of a medical device needing maintenance, prediction of complications in cases which may require future special care, or prediction of schedule delays. In exemplary embodiments, the streams of machine data from the medical devices may reflect the manner in which the medical device is being used and from analysis of the operation of the medical device, an event such as a complication during a surgery may be detected based upon this machine data. As will be described in further detail herein, the prediction of such a delay can be leveraged to reorganize schedules or room assignments for later scheduled procedures. In still further embodiments, the streaming analytics algorithms can be used to predict a likelihood of admission to an intensive care unit from an operating room or to predict a likelihood of avoiding the ICU after a procedure in the OR. These predictions may exemplarily be based upon the manners in which the medical devices were used during the procedure in the OR as a result from analysis from the streaming machine data from these medical devices.

In still further exemplary embodiments, the streaming analytics algorithms may be used to detect deviations from standard practice and/or to detect cases completing ahead of schedule or with a delayed schedule. For example, these may be detected by an identification of an unusually long induction phase, maintenance phase, or emergent phase as determined from the operation of an anesthesia delivery machine as compared to normal or expected operation. In other exemplary embodiments the results of streaming analytics algorithms may further be used to reduce nuisance alarms and to prioritize other alarms or patient critical alerts. Other exemplary embodiments apply streaming analytics algorithms to provide benchmarking of quality metrics for completion and provision of the procedures provided in the clinical case. Additionally, streaming analytics algorithms applied to the machine data of the medical device data can be used to infer likely activities or events during the procedure and document the times at which activities occurs for procedure summary and reporting as well as to predict post procedure outcomes.

The streaming analytics at 108 may classify events detected in the time series medical device data. These identified events may be classified as reactive, predictive, or prescriptive. Reactive events are those events which have already occurred. Predictive events are the events with statistically ranked outcomes and prescriptive events are those predictive events which also include recommended actions. At 110 deep learning algorithms may further classify the events with more specificity than the above noted reactive, predictive, and prescriptive. In non-limiting examples, the deep learning algorithms may classify the events as medical complications, likely admissions to the ICU, adverse outcomes, operational delays, high cost/waste of medical resources, additional events may be classified which detect early signs of trouble which can be both clinical and technical in nature. Additionally, the detection algorithms may flag high stress cases in addition to identifying chronology of medical device use, including delivery of various anesthesia phases, machine checkout and maintenance events, or the prolonged practice of high flow anesthesia. As will be explained in further detail with respect to FIG. 3, the behavior and accuracy of the predictions improve over time with more data, patterns, and through self learning.

The deep learning at 110 can be used to produce algorithms 112 for example through the methods as described in further detail herein with respect to FIG. 3. These algorithms may in turn be used for streaming analytics. Non-limiting examples of algorithms which may be developed from the deep learning at 110, may include algorithms to detect medical complications, to detect signs of clinical trouble, predict likely ICU admission, detect high flow anesthesia, detect high anesthesia cost or waste, detect high stress cases, detect signs of technical trouble in the medical device, or predict delays in further scheduled cases in a unit based upon detected prior events.

A case summary module 114 maintains state information for the clinical cases and tracks associated events for particular clinical cases. The case summary module 114 further helps the streaming analytics 108 by identifying recent historical events to help the streaming analytics 108 from identifying the same events excessively in the time series data and further maintains identified events in the chronological order. For example, if a clinical case start has been identified, focus can be redirected to identifying a clinical case end, rather than continuing to identify a further clinical case start.

In exemplary embodiments the streaming analytics at 108 can detect various events occurring in the streaming medical device data. Non-limiting examples of detected events may include those events which may be detected from a single data stream, for example a change in gas flow provided by an anesthesia delivery machine, for example a transition between high flow to low flow. An event that a machine checkout by a technician occurred may be detected, for example by noting a signal that is input or activated during a part of a checkout procedure. Various machine operability signals may be monitored, for example related to operation of a microprocessor to indicate whether or not the machine is with or without power or is in an on, off, or stand-by state. Respiratory events indicating patient airway troubles may be detected from the streaming machine data indicative of respiratory support provided to a patient in the cases of respiratory assistance for a spontaneously breathing patient or a clinician change to the respiratory support provided by a machine in the case of mechanical ventilation. Still further exemplary events which may be detected in the machine data include alarm status changes which indicate alerts and/or critical alarms, or technical alarms. In additional embodiments, events in the process of a medical procedure may be detected in the medical device machine data. For example, the start of a surgical case by detecting that medical devices are turned on or initialized, the detection of the start of the delivery of anesthesia or another medical device providing therapy or patient monitoring, detection of induction, maintenance or emergence phases, or the end of a medical procedure.

In still further exemplary embodiments, complex event processing may be used to identify events which occur upon event detection based not only upon the analyzed streaming medical device data, but also may be detected in view of the combination of multiple streams of medical device data and/or trends in various streams of medical device data.

Events detected by the streaming analytics at 108 are provided to an event router 116 which routes the identified events into predetermined groups or topics in an event notifier 118. The event notifier 118 receives the identified events from the event router into a plurality of groups or topics 120. The groups may be filled with similar events based upon type of medical procedure, type of device, location of device, or type of event. For example all of the events that are detected as originating from a particular hospital, or a particular OR may be grouped together for event notification. In another embodiment, all of the events related to a particular type of device e.g. an anesthesia delivery device, may be grouped together. In a still further example, any events detected related to anesthetic agent consumption may be grouped together. Within each group, the importance or severity of the events may be determined and a notification for the events produced accordingly. Low priority events may be stored to an event log for later review. While high priority events for example detected software or hardware malfunction, diagnostic or predictive events may be provided as disclosed herein to more directly alert responsible personnel.

A service bus 122 is connected to the event notifier and publishes the event notifications to the appropriate consuming endpoint depending upon the notification and the priority of the notification. Channels of transmission/communication of event notifications as well as personnel to which even notifications are communicated may be predetermined based upon the groups into which the event notifications are routed and/or services of the event notification. Notifications may be provided to workflow engines, for example as may be used to coordinate personnel and task workflow within a hospital at 124. Events may be directed to a push notification application programming interface (API) for sending and receiving push notifications to the identified recipient(s), for example on a wearable 128, a dashboard 130 on a laptop or desktop computer, or on a mobile device 132. In other embodiments, wearables, 128, dashboards 130, and/or mobile devices 132 may connect directly with the service bus 122, for example through an app or other program operating on the device that connects to the service bus 122.

FIG. 3 depicts an exemplary embodiment of a method 200 for data curation and deep learning. Exemplary embodiments of the method 200 may be carried out with the medical device data processing system 12 as described above with respect to FIG. 1. As depicted in FIG. 1 a plurality of medical devices 16 produce streaming time series of medical device data. The medical devices 16 may be located all within the same unit within a hospital, within the same hospital, or across a plurality of different hospitals and locations. The method 200 begins by continuously acquiring the streaming time series of medical device data at 202 from all of the medical devices. As noted above, the medical device data includes, but is not limited to data streams in time series format of machine data, for example alarms, device statuses, settings, messages, and data measured from device sensors. The medical device data may further include identified physiological data acquired by the medical device. These pluralities of streams of time series data from the plurality of medical devices are received by the medical device data processing system by ingesting the streams of data into the system to process the data for further processing and storage. At 206 the streaming analytics performed by the streaming processing engine 28 automatically detects clinical cases from the medical device data at 206. The clinical cases can be detected in the manners as described above. The identified clinical cases are further classified into normal cases and outlier cases. The identified clinical cases are saved, for example in the operational case database 30 and/or the data lake 26 with the summary of the case classification as normal or an outlier at 208. Edge cases and outlier cases are flagged at 210 for independent review by a clinician expert. The flagged cases are provided to clinician experts and are reviewed at 212 by the clinician expert. In the clinician expert review, the case summary of the identified clinical case is combined with the clinician's own manual case labeling at 214 and additional metadata 216 reviewed by the clinician in evaluating the flagged cases. The additional metadata reviewed at 216 may include the identified physiological information regarding the clinical case and/or events detected in that same clinical case. While at 218 the identified normal cases are submitted to the learning network at 220, the clinician expert has the option of submitting or withholding the reviewed edge or outlier cases from the learning network 220.

In a still further exemplary embodiment, identified clinical cases may further be evaluated and identified in a manner of case profiling. In exemplary embodiments, review and evaluation of clinical cases either automatedly or in a combination of clinician and automated review, can help to identify clinician work flows from the medical device data streams. For example, changes in anesthesia amounts during the course of a medical procedure can be identified and related to particular anesthesiologist techniques, work flows, or practices. For example, in one embodiment, anesthesiologist's use of anesthetic agent to control blood pressure may be identified while in other cases specific anesthesiologist's techniques or workflow, for example use of a “coasting maneuver” during an emergence phase may be detected.

Deep learning training 222 takes in new clinical cases and links the clinical cases and/or curated cases to the medical device data records in the data lake with each clinical case. The clinical cases and the associated medical device data are used by the deep learning network 220 to train deep learning algorithms 224. In embodiments, the deep learning network 22 may additionally incorporate curated data from a wide variety of clinical research institutions that have joined into a shared learning and discovering community. The deep learning algorithms 224 may be predictive algorithms and/or rules which define particular outcomes, predicted outcomes or other events. The deep learning algorithms 224 are quality tested at 226 against the clinical cases and medical device data representing those clinical cases as stored in the data lake of the system. The deep learning algorithms 224 can be provided to the stream processing engine for use in the streaming analytics as previously described. In exemplary embodiments, the streaming analytics 108 (FIG. 2), exemplarily by the stream processing engine 28 (FIG. 1) analysis of the machine data in order to identify ventilation mode usage, performance of recruitment maneuvers, use and level of PEEP, delivered tidal volume, as well as other measures of ventilation delivery and procedure effectiveness and use for example, oxygen saturation, hemodynamic values, end tidal gas concentrations, lung volume changes, and patient lung compliance. Relative values and/or changes in any of those metrics may also be used to identify between procedure phases.

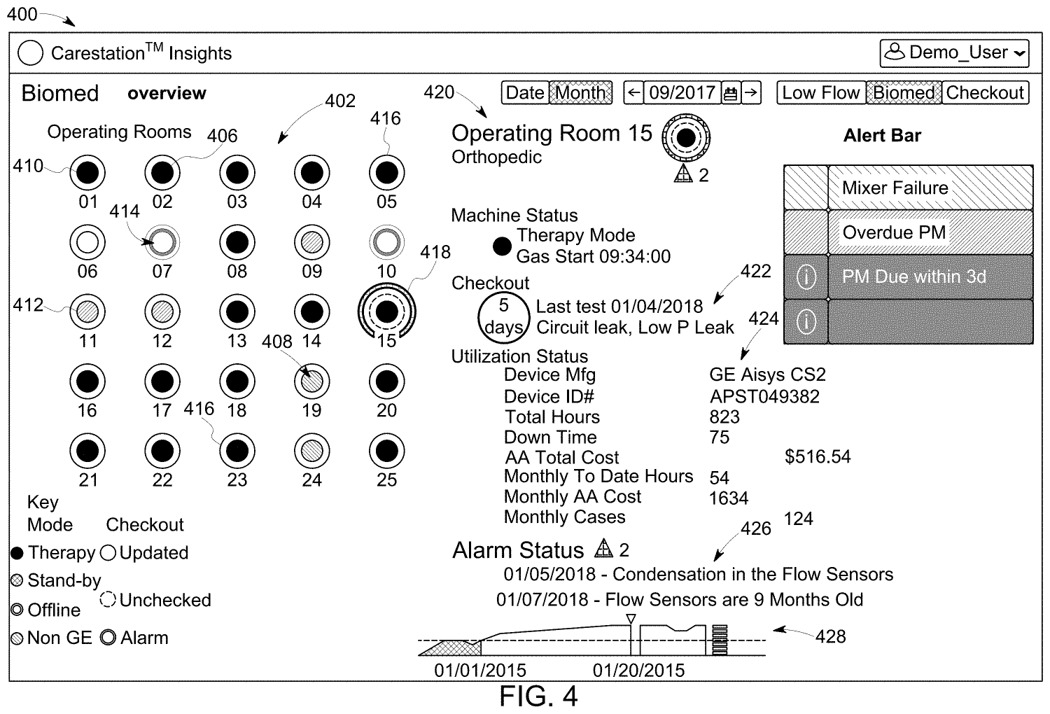

FIG. 4 depicts an exemplary embodiment of fleet management dashboard exemplarily in GUI 400. The dashboard presentation of medical device status and operation as presented and described herein can be provided in a local system as described above, or if the medical device data processing system is implemented remotely via a cloud computing solution, the dashboard can be provided back to the hospital either to report the locally obtained data or data across a plurality of hospital locations rather than a local implementation.

In a still further exemplary embodiment, the medical device data processing system and methods as disclosed herein may be implemented and carried out locally rather than in a cloud-based implementation. In a non-limiting embodiment, an anesthesia delivery management system 18 is locally connected to a plurality of anesthesia delivery machines 16b at a hospital 14 and receives the medical device data from each of the anesthesia delivery machines 16b. In a non-limiting example of such an embodiment, the anesthesia delivery management system 18 may carry out the medical equipment fleet maintenance management functions as described herein. The anesthesia delivery management system 18 may further perform the medical equipment fleet management analysis and reporting, exemplarily through a dashboard as depicted in FIG. 4. In a non-limiting exemplary embodiment, the streaming analytics may rely upon algorithms and/or rules supplied to the anesthesia delivery management system 18 rather than generated through a local deep learning process.

The exemplary embodiment of a fleet management dashboard 400 exemplarily provides a large amount of information regarding anesthesia delivery machines, or other pieces of medical equipment, including but not limited to patient monitors, across a fleet of devices for example in a surgical unit of a hospital. In exemplary embodiments, use of the dashboard 400, the other exemplary embodiments of the dashboard as disclosed herein, and the methods associated therewith which will also be discussed may be used by one or more hospital biomeds or clinical engineers tasked with troubleshooting, repair, and/or maintenance of the medical equipment of the hospital facility. In use, the hospital facility may be a network or complex of multiple buildings and/or locations. Effective management and prediction of medical device maintenance, as well as improved advance information regarding a technical problem can improve technician response time, effectiveness, and efficiency by managing the technician schedule and supplies before the technician is on location to inspect the piece of medical equipment. As will be provided in further detail herein, this management may include scheduling of the maintenance work within an equipment use schedule and prediction of the time to complete the scheduled work. The management may further include pre-identification of needed components, including consumables, such that the technician can arrive at the medical equipment to be serviced with the components needed to complete the maintenance.

In an operating room summary 402, each anesthesia delivery machine located in each of twenty-five exemplary operating rooms is represented. A key 404 presents a graphical output of machine information and status whereby a center circle identifies a GE unit (solid color) 406 or a not-GE manufactured product as exemplarily indicated by hatched lines 408. This information can be pre-established from a database or manually identified when adding the monitored device to the fleet management dashboard 400. In a still further embodiment, the identification and/or make and model of the monitored device can be identified in an encoded signal provided as part of the MDD or determined from the MDD values, for example in the form or format of the MDD data provided which may be unique to a particular make or model of device.

The operational status of each of the anesthesia delivery machines is further indicated with e.g. green colored solid circle 410 indicating an anesthesia delivery machine in use to provide therapy while an e.g. orange colored solid circle 412 indicates an anesthesia delivery machine in a standby mode and an empty center circle 414 indicates an anesthesia delivery machine that is currently offline. Such current machine status identifications are known within the system through the analysis of the MDD as described in further detail herein. Additional rings located around the center circle further indicate the checkout status as discussed above. Separate colors of intermediate rings 416 can indicate whether the anesthesia delivery machine has successfully passed the appropriate checkouts, requires checkout updating, has gone unchecked, or has missed a scheduled checkout. Finally, an outer ring 418 can indicate an alarm detected for each anesthesia delivery machine. It should be appreciated that other embodiments of the legend and coding may be envisioned and are within the scope of this disclosure. As noted above, this information can all be obtained through the data streams of machine data collected from the anesthesia delivery machines or other medial equipment in the monitored surgical unit.

By way of example, the current operational status of each of the anesthesia delivery machines between offline, standby, or therapy modes can be determined from the streams of machine data as described herein. Similarly, the checkout status can also be determined. In an exemplary embodiment, the streaming MDD may include an indication when a most recent checkout procedure has been performed at the medical device. In other exemplary embodiments, the streaming MDD may include keystroke data from the medical device. This keystroke data can be analyzed to detect the performance of a checkout procedure, for example by recognizing a keystroke sequences in the streaming MDD that are indicative of a checkout procedure. In a still further exemplary embodiment, the checkout procedure, due to the order of operations and the output MDD as the machine performs the procedure, the MDD processing system may identify the occurrence of the checkout procedure from the MDD. In such an embodiment, individual systems may be turned on and tested. The test may produce an expected, output and output duration, particularly if the medical equipment properly completes the checkout procedure. Due to the operational values and/or the timing duration and sequence of system operations may indicate that the medical equipment is not being used in a surgical procedure. Such indications of a checkout procedure may be applied in connection with, or with knowledge of, a scheduled checkout or with a current use status as described herein to determine if the machine is checked or unchecked. In an embodiment wherein any alarm signals are provided in the machine data, the reported alarms status can be determined in this way as well.

Selection of any one of the anesthesia delivery machines in an exemplary operating room can pull up a detailed machine report 420. Exemplarily as found in FIG. 4 the machine report 420 can provide a current machine status indicating the mode in which the anesthesia delivery machine is currently operating, and also an indication of when the anesthesia delivery machine last changed status. A checkout report 422 provides a summary of information as may have so been presented in a checkout dashboard, indicating a checkout status, need for checkout test, and/or a result of a most recent checkout test.

A device utilization status 424 reports both identifying information regarding the particular anesthesia delivery machine as well as reports regarding the machine use. Information regarding machine use can be kept track of by analysis of the streams of machine data to identify total hours of use in a therapy mode, total hours in a standby mode, and utilization regarding numbers of cases and anesthetic agent delivered with the machine. In still further exemplary embodiments, some medical devices may be operated in different therapeutic modes. In a non-limiting embodiment, some anesthesia delivery machines may be optionally operated as a mechanical ventilator without delivery of anesthetic agent. A utilization status 424 may further breakdown in the modes of use of that particular medical device. For example, this may be detected in a comparative manner based upon the streams of machine data related to both anesthetic agent delivery and the provision of respiratory support. Detection of operation of a machine to provide respiratory support without the delivery of anesthetic agent can be an indication that the medical device is being used to provide only respiratory support while in other uses, the same machine may be used to only deliver anesthetic agent or to deliver both anesthetic agent and respiratory support. In still further exemplary embodiments, a machine status provided in a stream of machine data from the anesthesia delivery machine may indicate whether the machine is operating in a anesthesia delivery, respiratory support, or combinational function. In an example, this information may also be provided to the business intelligence and visual analytics tools 32 as previously described to present insight into a clinical use and operation of medical devices. From this information, the actual use of various systems of the medical equipment can be monitored. As explained herein, actual use measurements can provide for a more economical deployment of maintenance and replacement of consumable and other replaceable parts.

An alarm status 426 can provide a more detailed explanation of the detected alarms. These detected alarms may exemplarily be technical alarms, for example detection of condensation on the flow sensors, or an excessive age of flow sensors as indicated in the example of FIG. 4, although the alarms are not herein so limited to only technical alarms.

The dashboard 400 may further present a graph 428 of fleet performance over time. In exemplary embodiments, the fleet performance may be measured and reported in a variety of ways. Fleet performance may be represented as a fleet utilization rate, or the percentage of time that each device is used during each day. The utilization rate may alternatively be reported as a number of device-hours used each day. In another embodiment the fleet performance may be represented as the percentage or number of devices that are currently deemed operational and without maintenance issues. In still further embodiments, the fleet performance may be a composite metric that weights some or all of the above-noted metrics, still other metrics may be incorporated into such a metric, including but not limited to current or recent maintenance or technical alarms.

In an exemplary embodiment, the anesthesia delivery management system 18 may calculate a vapor flow rate for each anesthesia delivery machine 16b as it is used during an identified surgical case. This calculation may be based upon information obtained and identified from machine data from each of the anesthesia delivery machines 16b. A stream processing engine operating, for example, on the anesthesia delivery management system 18 can detect the event of a surgical case beginning through the anesthesia delivery machine data time series streams, for example which may show the anesthesia machine powering up, going through an initial checkout process or cycle, and then the initiation of delivery of anesthesia by the machine. This may be used by the stream processing engine to identify the clinical event of a surgery using anesthesia. Additional medical device data streams from that anesthesia delivery machine or other medical equipment, e.g. a patient monitor, associated with that anesthesia delivery machine may also be used to identify the clinical event. The stream processing engine 28 may further receive a calculated anesthetic vapor flow rate from the anesthesia delivery management system 18, or may receive the individual device settings of the vaporizer to enable calculation of anesthetic vapor flow rate. Anesthetic vapor flow rate can be calculated based upon a fraction of inspired agent and the fresh gas flow rate. Still further embodiments may additionally require the expired minute volume the inspired minute volume and a fraction expired anesthetic agent.

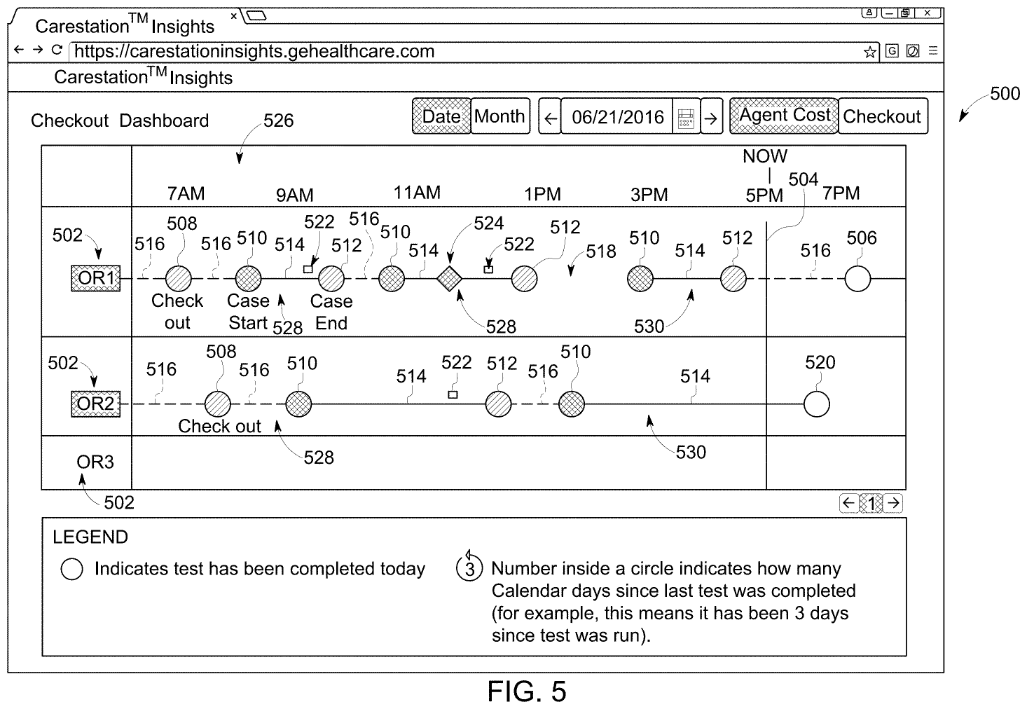

FIG. 5 depicts a further exemplary embodiment of a monitored room status dashboard presented as GUI 500. In the GUI 500, a temporal view of the use each of a plurality of monitored rooms 502 is reported to visualize both a current status of the use of an operating room, but also a future schedule of the operating room, this visualization can assist a clinician or clinical manager in scheduling of maintenance or repair of medical equipment. The calculated information presented in these views can further be used to determine need and scheduling of medical device components and routine maintenance based upon medical device use and component performance.

The GUI 500 exemplarily presents a temporal presentation with a line 504 indicating a current time. Future scheduled events 506 are presented to the right of the line 504 while the actual timing of the procedures as they occurred are presented to the left. The events of the procedures as they occurred can be detected by analysis of one or more streams of medical device data, including streams of machine data from multiple medical devices located in each of the monitored rooms 502. The analysis of machine data from multiple medical devices associated with a medical procedure can further improve accuracy of event detection by the correlated indications found in the machine data of these separate devices. In an exemplary embodiment, individual circles may indicate when checkout procedures 508 have been performed. Connected circles indicate the start 510 and ends 512 of various procedures. The operational status of machines or events may be indicated by a line. Solid lines 514 indicate a procedure while dashed lines 516 indicate a standby status. A gap or no line 518 may indicate when machines are off or a facility is otherwise closed. In an exemplary embodiment, when a procedure is currently ongoing, the system may produce an estimation of a procedure end time 520. This estimation of procedure end time 520 may be exemplarily based upon the underlying schedule as well as any detected events in the medical device data during the procedure thus far that would indicate a delay in the estimated procedure end time. Detected events 522 may be indicated along the procedure which may indicate a prolonged procedure, or other departure from a baseline estimate of a procedure end time. Still further events, for example a secondary induction phase 524 may be indicated as such an event may further indicate a prolonged procedure. In exemplary embodiments, medical devices may be designed to incorporate additional sensors such as to further collect and report machine data as used in exemplary embodiments described herein. Such machine data may exemplary include flow sensor or valve cycles, processor clock speeds, component voltages. Further streaming time series MDD may include indications of when particular components (e.g. battery, humidifier, CO2 absorber) are in use. In still further exemplary embodiments, streaming time series MDD that includes the measured parameters from the operation of the medical device, for example system pressure, volumetric flow rates, or measured expired gas concentrations are used as described herein to evaluate medical device component performance. Changes in these signals may exemplarily be used to track a condition and/or “wearing out” of consumables and/or commonly replaced components of a medical device, non-limiting exemplary embodiments may include a CO₂ absorber, humidifier, filters, valves, or gas sensors.

The GUI 500 includes a table 526 that presents an overview of the past and current status of each of a plurality of monitored rooms 502, e.g. operating rooms. Each monitored room 502 is represented in a row in the table 526. The table 526 provides a temporal depiction of the status of the monitored rooms 502 and exemplarily provide a visual depiction of concepts as may be used herein to determine maintenance needs of medical device components and/or to schedule required maintenance in a manner that reduces medical device operational down time.

Relative to the current time 504, the table 526 presents indications of past procedures 28. The ends of the indications of past procedures 528 can coincide with a detected procedure start and/or completion times and the lengths thereof thus representative of procedure length. The identified past procedures 528 provide a record of the prior use status of the monitored medical devices. As will be described in further detail herein, detected actual use of the monitored medical devices can be used to derive more accurate and economical schedules of medical device maintenance or consumable replacement.

The table 526 further identifies active procedures 530. The active procedures 530 may be indicated in one or more colors or visual effects, for example to set them apart from the indications of past procedures 528. As described in further detail herein, the system may further operate to detect particular events or phases within the active procedure 530. For a patient receiving anesthesia, the anesthesia status of the patient may be determined to be in one of three anesthesia phases—induction phase, maintenance phase, and emergence phase. The start, end, and length of the phases further exemplarily coincide with the time identifications of the table 526. The identification and temporal length of the operational phases of the medical devices can be used in two exemplary ways to manage medical device maintenance. First the temporal length can be used to track the actual operational use of the medical device components. For example anesthetic agent is delivered to the patient during an induction and a maintenance phase, therefore, those components are actively in use for the durations of those detected phases. Secondly, the detected phases of the active procedure can be used to predict when the active procedure will be completed and maintenance can be performed with minimized down time of the medical devices between procedures.

As noted above, embodiments of the system operate to analyze MDD, for example taken from the anesthesia delivery device itself, as a source of de-identified data that can be refreshed and ingested at a high temporal resolution to provide both a real-time indication of the current use status of one or more operating rooms, but also for prediction of when future use status will occur within each monitored room. Such use status indications and/or predictions of future use status occurrences may require analysis of multiple streams of MDD. The current use status, procedure phases, or predictions of the occurrence times of future use statuses or procedure phases can be performed as described herein through analysis of the streaming time series MDD without further user or clinician input, and may also operate without input from other scheduling tools. However, as will be described herein, the output of such determinations may be inputs to available scheduling tools. In still further embodiments, the time duration for each procedure phase may be determined and used as an indicator or in a determination of when a subsequent procedure phase will begin or when the current use status of a monitored room will change.

MDD from the anesthesia delivery device can include values that are indicative of the presence of patient exhalation (e.g. exhalation pressure, tidal volume, expired gas concentration). MDD from the anesthesia delivery device may also be indicative of system operation, for example system pressure. In exemplary embodiments as described in further detail herein, the system pressure or other values indicative of system operation may be used to determine leaks in the system or to evaluate the operation of the CO2 absorber, humidifier, gas sensor(s), or filters. In other embodiments, signals indicative of when the battery is in use and/or the remaining power in the battery may be used. In still further embodiments, the MDD may include an identification of any alarms that occur and/or a transmission of any alarms, errors, or notifications that are electronically logged by the medical device.

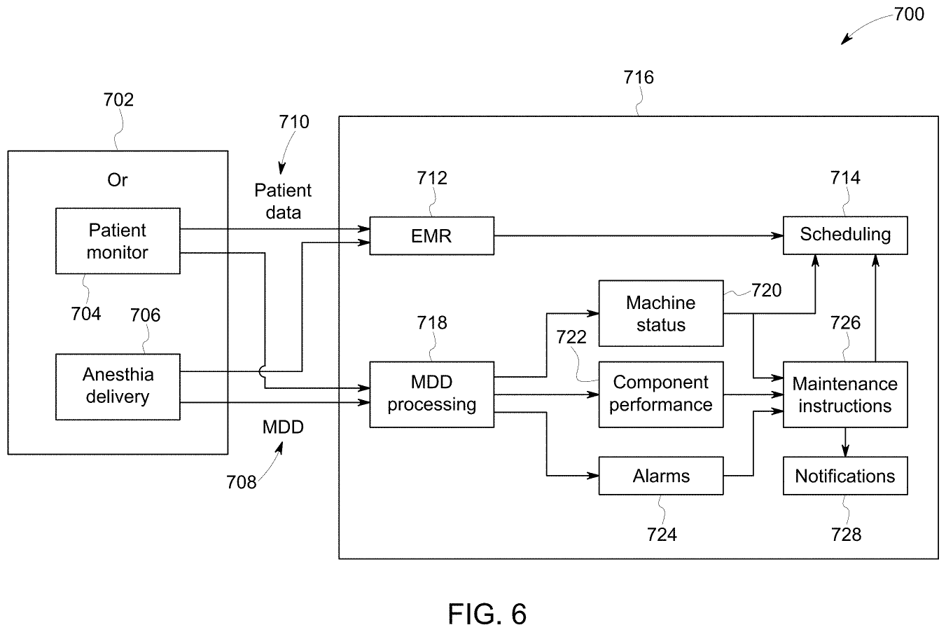

FIG. 6 depicts an exemplary embodiment of a system 700 for processing medical device data (MDD). The system 700 depicted in FIG. 6 is exemplarily provided in two portions. A local portion 702 occurring within an operating room (OR) or within another monitored room as described. While the example of an operating room is used herein, it will be recognized that in other embodiments, the local portion 702 may take place in another type of monitored room within a medical care facility, provided that such monitored room includes at least one medical device that produces MDD as described in the present application. The monitored room of the local portion 702 includes a medical device of a patient monitor 704 and a medical device of an anesthesia delivery machine 706. It will be recognized that other medical devices may be used within the monitored room 702 and thus may operate in the manner as described herein while remaining within the scope of the present disclosure. One or both of patient monitors 704 and the anesthesia delivery machine 706 produce medical device data (MDD) as described above. The MDD 708 from patient data 710 which may also be produced by one or both of the patient monitor 704 or anesthesia delivery machine 706 in that the MDD 708 is de-identified in that the streaming time series of MDD is produced and provided without identifying information which may relate to the specific patient, but rather to the function and operation of the machine from which the MDD is provided. The patient data 710 in contrast includes identifying information of the patient and thus is provided in a secure manner to an electronic medical record (EMR) 712 and provide either in series or in parallel from the EMR 712 to a medical facility scheduling system 714.

The local portion 702 is distinguished from the processing portion 716 of the system 700. The processing portion may be implemented in a cloud based system or may be implemented locally to the specific monitored room. Still further embodiments may be implemented in various manners between these two embodiments, for example with processing remote from the monitored room, but at another location within the medical facility, or in a distributed processing network in which portions of the processing may be performed on different devices and locations.

The MDD 708 is provided to an MDD processing system 718 as previously described. It will be recognized that the MDD processing system 718 may be implemented on one or more computers and may include the features as described above or more or fewer features based upon that disclosure as will be recognized by a person of ordinary skill in the art. The MDD processing system 718 receives streaming time series of MDD from at least one medical device in one or more monitored rooms. The MDD processing system 718 analyzes these streaming time series of medical device data to detect procedural events in the time series MDD. Procedural events may be detected as described above, and may be done so based upon models of expected MDD values or occurrences within a modeled or expected procedure.

For example, based upon the time series MDD, medical device start up, initialization, and set up procedures can be detected and noted to identify the start of a procedure. Once a procedure is started, the use status of the monitored room can be identified and a current phase of the use of the monitored room identified. With regularly defined phases to detect procedures in a monitored room, the progression and/or occurrence of a phase can be determined from the time series MDD, for example based upon gas flows or particular operations or settings performed on the one or more medical devices in the monitored room. The MDD processing system 718 thus provides indications of the use status of each of the monitored medical devices at 720. These indications may include a current use status of each medical device, including whether the machine is in use or not in use while further status indications may include a procedure phase of a current use.

Additionally, the MDD processing system 718 can operate to identify the streaming time series of MDD that relate to or are otherwise indicative of performance of particular components in the monitored device and further use these identified streaming time series of MDD to determine component performance 722. The determination of component performance 722 exemplarily is performed by the MDD processing system 718 as well and is based upon comparing models representative of particular component break down or wear to the streaming time series of MDD. The component performance 722 may be determined by a comparison to other MDD of historical operations of the same or other monitored devices, while in other embodiments, the current MDD may be compared to a model based upon historical MDD, which reflect a normal or properly functioning component. Such a model may comprise multiple series of MDD that relate to one another in depicting the performance of a component or system. In exemplary embodiments, the component performance 722 may identify a degradation in the MDD values over time, indicating that a related component is wearing out. In one embodiment, the system pressure may be lower than expected or may slowly drift lower during use. In such embodiments, this may be indicative of the development of system leaks in the breathing circuit. In another embodiment, MDD may be provided that is indicative of a remaining charge on a battery. If the battery is draining charge at a faster than expected rate, then this may indicative of the battery needing to be replaced. In a still further exemplary embodiment, an increase in patient inspired faction of CO2 may be indicative of the Carbon Dioxide absorbent material needing to be replaced. In still further exemplary embodiments, differences between expected gas concentration values and measured gas concentration values may provide an indication that a gas sensor needs to be replaced.

The MDD processing system 718 can further operate to receive and identify alarms in the streaming time series of MDD. The streaming time series of MDD can include a stream of indications of any alarms, or events or notifications as would be documented to an event log of the monitored device. The alarm analysis 724 may be performed by the MDD processing system 718 as well and may further be carried out by comparing rules as previously determined in the processes described above to relate alarms, events, or notifications from the streaming time series MDD to corrective maintenance procedures required for the monitored medical device. Such rules may identify from learning in the manners as described above, that some alarms do not have an underlying maintenance requirement, while in other instances, patterns or combinations of particular alarms, events, or notifications may be indicative of a need for particular maintenance.

Translation of alarms, events, or notifications into maintenance requirements can be a challenge as the alarms are typically crafted or reported with a clinician as the intended audience and is reflective of an observed effect in the system, but does not necessarily provide much insight into any maintenance issue that could have resulted in the observed alarm condition. Therefore, by way of the MDD processing system as described above with respect to FIG. 3, alarm data can be analyzed and related to maintenance tasks and outcomes to produce alarm interpretation rules. The alarm interpretation rules may interpret a plurality of alarms, alarm sequences, and/or alarm frequencies into a predicted maintenance task. In the event that two or more possible predicted maintenance tasks are identified, clarifying procedures or troubleshooting operations can be identified based upon the multiple possible predicted maintenance tasks. These procedures or troubleshooting operations can be based upon an efficient resolution to the maintenance issue and/or ruling in or out of possible predicted maintenance tasks. Based upon the predicted maintenance task or possible predicted maintenance tasks, the technician can be informed of a parts list based upon a likelihood of a particular part or piece of equipment being needed for the maintenance task. In this manner, the technician can arrive locally at the monitored medical equipment with the most likely needed tools, equipment, and parts for the predicted maintenance task.

In still further embodiments, combinations of identified machine status 720, component performance 722, and alarms 724 may be used by the MDD processing 718 to determine a maintenance requirement. For example, analysis of the machine status 720 may be combined with measures of component performance 722 in order to determine that a component needs to be replaced. Degraded component performance at 722 in combination with a known duration of actual machine use or component use from the machine status 720 may provide a more robust determination of required maintenance. Additionally, a component performance 722 may have different expected operations, functions or values during different phase of the use of the monitored device, and coordination between the operation of a particular component and the operational use phase of the monitored device may more accurately reflect maintenance need. In another exemplary embodiment, alarms may be combined with current machine status and/or component performance to determine maintenance needs.

The analysis of the machine status 720, component performance 722, and alarms 724 are used by the MDD processing system 718 to produce maintenance instructions 726. The maintenance instructions at 726 may be determinations to replace a component, for example a battery, a CO2 Absorber (or CO2 absorbent material), a humidifier, a gas sensor, a filter, or breathing circuit leak detection. In still further embodiments, an intelligent decision regarding routine maintenance, calibration, or check outs may be made based upon an actual time of use or time of use phase duration. These maintenance instructions 726 may be provided as notifications 728 which may be audible, textual, or graphical. In exemplary embodiments, the notifications 728 may be presented in the dashboard GUI interfaces as described herein. In other embodiments, the notifications 728 may be electronic alerts, to workstation, mobile, or wearable devices, for example in the form of text messages, push notifications, or in-app notifications.

In still further embodiments, the indications of machine status at 720 can be used in various ways, including as an independent real-time feedback input to the scheduling system 714. Because the monitored room status is determined from de-identified machine data, such real-time indications of monitored room status may be used to update a broader range of scheduling or notification functions, including, but not limited to, scheduling of cleaning teams, maintenance or service personnel, or inspection/management checks. Additionally, the indications of monitored machine status 720 may further include a predication of when either a current phase of a current procedure will end, or when the current procedure will end. Such estimation may be based upon modeling of records of previous similar procedures and generalizations from prior similar procedures, for example from detected events, values, or occurrences in the time series MDD. These estimations of the end or commencement of the current or a subsequent procedure phase or use status can be similarly provided to the scheduling system 714 for use in scheduling of when to perform the identified maintenance 726.

In a still further exemplary embodiment, the medical device data processing system and method as disclosed herein may be implemented and carried out locally rather than in a cloud-based implementation. In such an embodiment, for example, the use status determination methods and dashboard as described herein may be carried out for example at the anesthesia delivery management system 18 (FIG. 1). In a non-limiting example of such an embodiment, the anesthesia delivery management system 18 may carry out the clinical case identification, event detection, and maintenance determination processes described above. The anesthesia delivery management system 18 may further perform the procedural phase and phase/use status completion or initiation estimates locally to each monitored room. In a non-limiting exemplary embodiment, the streaming analytics may rely on algorithms and/or rules supplied to the anesthesia management system 18 rather than relying upon a locally implemented deep learning process.

In other exemplary embodiments, the use status, current procedure phase, completion/initiation estimations, consumable replacements, and maintenance determination may be embodied in a plurality of rules and/or processes which may be compared to the detected clinical events or other of the streaming time series MDD, as described above.

The systems and methods of processing MDD as described herein exemplarily provide further analysis of device maintenance. In exemplary embodiments, the MDD processing can be used to provide an improved automated diagnosis of device maintenance or device consumable replacement needs. This information can be provided to a centralized location, for example as reported on the fleet management dashboard as previously described herein to provide maintenance alerts, but also guidance for the required maintenance such that the maintenance can be accurately scheduled and the correct equipment or parts brought to the maintenance call. In additional embodiments, by accurately determining the type of maintenance needed, such maintenance can be scheduled to occur between scheduled between surgical procedures and an estimate of the maintenance time can be incorporated into the schedule.

The streaming time series of MDD can include either a direct indication that a checkout procedure has been performed, or the MDD processing system can apply detection rules to identify in the MDD if the checkout procedure has been performed. This can either be detected in keystroke logging provided in the MDD or may be detected by the MDD processing system by identification of particular test settings or routines, for example gas pressures, flow rates, or operation of particular components. Timing and duration of component operations may also be indicative of a checkout procedure, which may be detected similarly to that of other procedural events as described above. In addition to identification of a checkout procedure, the MDD processing system can further evaluate the MDD resulting from the checkout procedure to determine if the checkout procedure is successful, or if the checkout procedure is unsuccessful, then which component failed the checkout or which portion of the checkout procedure was not completed.

In exemplary embodiments, the MDD can include operational or keystroke data. This can be stored in a log at the MDD processing system. In the event that a technical alarm or other event or notification occurs, further analysis of the operational or keystroke data at the time prior to the alarm, event, or notification. Additional analysis of this keystroke data can provide information regarding the interaction between a clinician and the machine just prior to the alarm, event, or notification to be corrected. This context may help to improve the determination of the maintenance recommendations provided by the system.

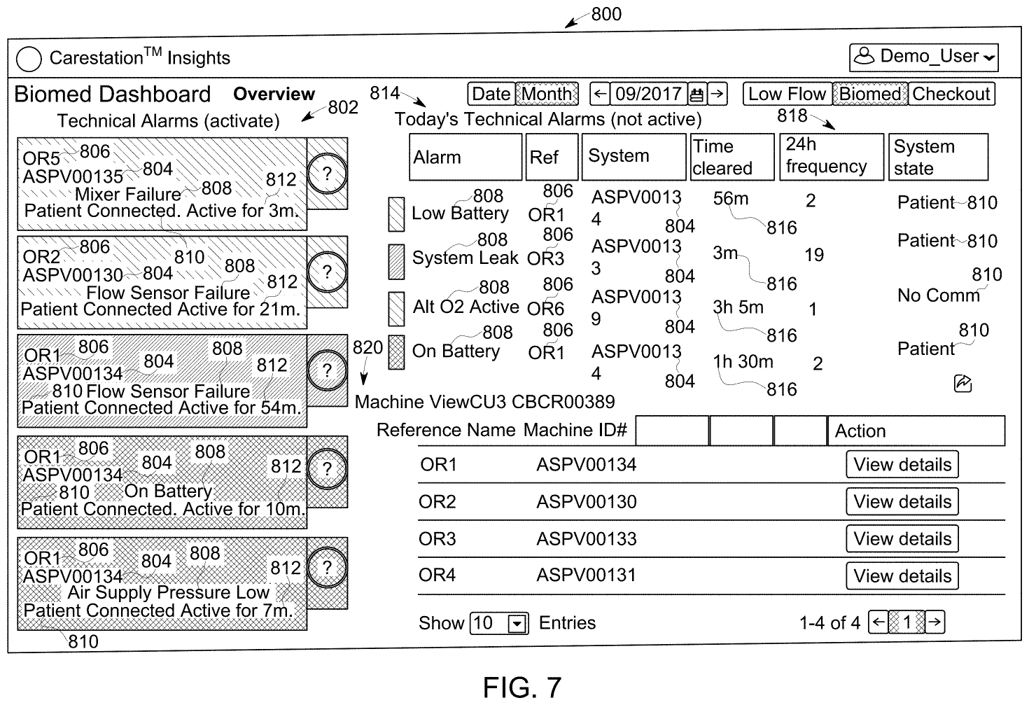

FIG. 7 depicts an exemplary embodiment of a fleet management dashboard exemplarily in GUI 800. The GUI 800 of the fleet management dashboard may be used in connection with the GUI 400 described above with respect to FIG. 4. The GUI 800 specifically presents examples of technical alarms that may be experienced during use or operation of the system. Active technical alarms 802 are presented exemplarily with a color code to indicate severity. The list of active technical alarms, exemplarily identifies the machine 804, the location of the machine 806, the technical alarm name 808, the status of the machine (e.g. patient connected, standby, etc.) 810, and the duration of the alarm 812.

The GUI 800 further presents a table of inactive alarms 814. The table of inactive alarms 814 provides the same information as the identification of active alarms 802, but also provides the amount of time required to clear the alarm and a recurrence count 818 of the number of times that that alarm had occurred in the past twenty-four hours. Lastly, the GUI 800 provides the option to select by machine or location at 820, which may present a view as described above with respect to the machine report 420 in FIG. 4.

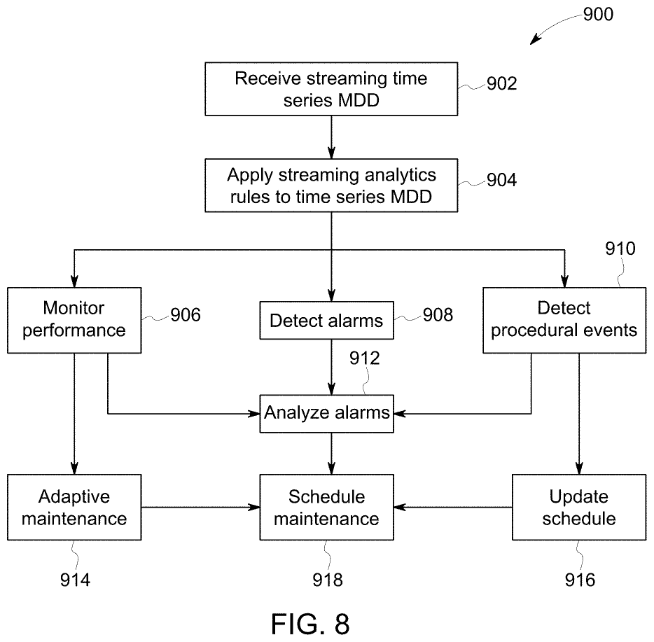

FIG. 8 is a flow chart that depicts an exemplary embodiment of a method 900 of managing maintenance of medical devices. The method 900 begins with receiving streaming time series medical device data (MDD) from a plurality of monitored medical devices at 902. As previously noted, the time series MDD includes de-identified information and values from the medical device regarding the operation of the medical device. Such streaming time series MDD may include medical device input or settings or the values read by sensors incorporated with the medical device. In an exemplary embodiment, the time series MDD includes at least one, some, or all of the following MDD: alarms, event or notification logs, keystroke/input logs, fresh gas or component gas flow rate(s), anesthetic agent concentration delivered or exhaled, inspired CO2 concentration, CO2 exhalations by the patient, ventilation system pressure, ventilation system flow, delivered volume, breath rate, expired gas concentrations, battery usage, and/or remaining battery life.

Next, at 904 streaming analytics rules are applied to the time series MDD. The streaming analytics rules applied at 904 may be a predefined rule set or may be rules that are derived from the machine learning processes as described above. The streaming analytics rules can identify patterns in the streaming time series MDD in manners such as to perform one or more of three different identifications. The streaming analytics rules at 904 can monitor machine performance at 906, detect alarms at 908, and detect procedural events at 910. These can be performed individually, or may be used in combination to provide improved maintenance management across a plurality of medical devices. In one embodiment, the types of identifications used for the determined maintenance may depend upon the types of identifications found in the streaming time series MDD from the application of the streaming analytics rules to the time series MDD at 904.

At 906 the streaming time series MDD yields identifications of MDD that relates to component performance. By trending these identified MDD over time, the performance of particular components, particularly replaceable or consumable components, like CO2 absorbent material, batteries, filters, humidifiers, gas sensors, or breathing circuits, can be evaluated and performance degradation identified or replacement predicted. In non-limiting examples, CO2 absorbent material may be evaluated by monitoring the inspired CO2 concentration. In other embodiments, the breathing circuit may be evaluated based upon the measured flow rates or pressure within the system. In still further embodiments, the actual use time of a particular component may be identified and evaluated. The actual use time, or characterizations of the use time may be more predictive in estimating a need for component replacement than simple reliance upon a replacement schedule based upon component averages.

At 906 the streaming time series MDD yields identifications of alarms. This may also include counting/aggregating of the identified alarms and determination of the frequency of the identified alarms over a relevant time period. The alarms may be provided as a feed of the actual technical alarms from the monitored medical device. In such an embodiment, the streaming analytics identifies each particular alarm and provides any segmentation or cataloging of the alarms as reported in the streaming analytics. In another embodiment, the technical alarm rules are independently applied by the MDD processing system to the streaming time series of MDD. In such an embodiment, the streaming analytics rules applied at 904 performs an independent evaluation of the existence of any technical alarms in the MDD.

Next at 910, procedural events are detected in the streaming time series MDD. The procedural events detected in the MDD may include changes in medical device operation and which may be indicative of a current use or operation of the medical device. Such events may include the initialization or set up procedure of a medical device, a change or initiation in gas flow rates, a change in system pressure for example due to connection or disconnection of the patient from a ventilation system, delivery or weaning of anesthetic agent concentrations, an oxygen flush, or other medical device operations. Still other examples of procedural events include procedure start and stop times, and/or monitored medical device start and stop times. The procedural events may further include actual use time of particular monitored medical devices and/or components of monitored medical devices.

One challenge for maintenance guidance for monitored medical devices is to connect technical alarms to the maintenance tasks required. At 912 any alarms detected in the MDD are analyzed to determine the associated maintenance tasks that are required. This analysis may further include alarm frequency, duration of alarm occurrence, and combinations of alarms occurring at the same time. In performing this analysis at 912, the monitored performance 906 of any devices and/or components, as well as the detected procedural events at 910 are used in combination with the detected alarms 908 in order to determine the maintenance tasks required to address the technical alarm.

Furthermore, the component performance monitored at 906 may lead to adaptive maintenance recommendations at 914. The adaptive maintenance may instead use component performance or actual use times to determine servicing or replacement as opposed to a temporal schedule or routine maintenance or replacement. Also, the procedural events detected at 910 can be used at 916 in order to update the current schedule and any prediction of when a procedure will be complete and a room and/or medical device out of use. With improved and updated scheduling, including prediction of event completions at 916, the maintenance can be scheduled at 918 in a manner so as to minimize disruption to the schedule. In still further exemplary embodiments, with improved maintenance recommendations, a more accurate estimate of the duration of the maintenance procedure can me made to further improve the coordination of the maintenance tasks scheduled within the procedure schedule.

In exemplary embodiments, determined maintenance and maintenance scheduling as described above and as would be recognized by a person of ordinary skill in the art in view of the above disclosure may be provided to the system operating to perform the method 300 and exemplarily as further used in the above-disclosed streaming analytics as previously hard coded (or manually coded) rules. These rules may be provided upon an initialization or set up of the system operating to perform the method 300, or may exemplarily be provided or updated through a software update to the system or a portion thereof. In other embodiments, the use status, procedure phase, and timing estimates and/or other rules used in the streaming analytics may be developed and provided by machine learning algorithms that develop the use status, procedure phase, and timing estimates and/or other streaming analytics rules through training and/or analysis of case data as described above.

As previously discussed, improved streaming analytics algorithms, for example for event detection, parameter value detection, component performance monitoring, or maintenance task identification may be created over time through the use of deep learning analysis of the machine data and patient data regarding use status, procedure phase, and timing estimates in the manner as described above. Additionally, such techniques may also be used to improve the algorithms and/or analysis performed to provide improved determinations of use status, procedure phase, and timing estimates.

In exemplary embodiments, analysis of machine operational status (e.g. in use, standby status, and offline) may further provide information and insights not only to a hospital to assist in efficient management of medical device assets, but may further provide generalized information regarding specific use case and use trends to provide insight into field use of medical devices, in still further exemplary embodiments, maintenance, use, or other schedules may be based upon utilization reports specific to particular devices.

In a still further exemplary embodiment, the analysis of the streams of medical device data may further facilitate clinical record keeping and report management for a particular medical procedure. In an exemplary embodiment, analysis of the machine data can yield a summary report that indicates the time of use, duration of use, anesthetic agent delivered, as well as a log of anesthesia delivery events, inputs or changes in the anesthesia delivery. As this information can be detected in the streams of medical device data, such a report and/or summary can be automatedly produced and electronically provided to a clinician for review and approval and/or revision and the annotation with additional comments. In an exemplary embodiment, the use status, procedure phase, and timing estimates may be associated to medical billing codes and a provisional medical billing report provided to the clinician or another hospital personnel for review and documentation or use in billing submissions. In an exemplary embodiment, the electronic case summary report may be expressed in the HL7 clinical document architecture standard for example to be incorporated into a patient's electronic medical record and/or to facilitate use for billing documentation.

In an exemplary embodiment, the streaming analytics of the time series medical device data can include a combined analysis of machine data and physiological data related to the same patient in real time or near real time. Upon evaluation of this combined information the patient condition and current device settings and operation can be determined. Based upon this determination, particular events may be identified, these events being recommended changes to medical device settings or parameters. This may include adjustments to any of the values in the received machine data or other identified medical device settings, including alarm parameters. In one exemplary embodiment, these events result in notifications to a responsible clinician with recommendations for such medical device setting changes. In other exemplary embodiments, the medical device data processing system communicates back to the medical devices, for example through the gateway 20 to provide operational instructions and/or medical device setting adjustments directly to the medical device. In a still further exemplary embodiment, such recommended actions may first be presented to a clinical or technician for approval and/or confirmation prior to the MDD processing system providing such instructions directly to one or more medical devices.

Exemplary embodiments of the systems and methods as described herein gather machine data produced by a plurality of medical devices in real time and at a high frequency approaching the frequency at which such machine data is produced by the medical devices. Streaming analytics of this machine data can yield large amounts of machine use and medical procedure data that, in exemplary embodiments is, by its nature de-identified. This procedure data can be summarized for improved retrieval and access at a later date, but stored in its raw form for use in data analytics and machine learning to automatedly produce are and improved algorithms exemplarily for use in the streaming analytics of the medical device data.

Embodiments of the systems and methods as described herein improve upon existing health information systems/electronic health records data as that data is typically focused on patient physiological data and reported and/or recorded at longer time intervals. The low frequency of this data limits the types of clinical decision support that can results from these sources of data. For example reduced data resolution can lead to inaccuracy in detection of minimum and maximum values and transient events/values in the collected data. Furthermore, the use of patient physiological data introduces concerns and/or challenges of maintaining such information in a confidential and/or de-identified state.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives and modifications are possible within the scope of the appended claims.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of managing maintenance for a plurality of monitored medical devices, the method comprising:
receiving streaming time series medical device data from the plurality of monitored medical devices;
analyzing the streaming time series medical device data to determine an operational status of a component of a medical device of the plurality of monitored medical devices;
determining a maintenance procedure for the medical device from the operational status of the component of the medical device; and
producing a notification of the determined maintenance procedure.

2. The method of claim 1, wherein the streaming time series medical device data comprises alarm data.

3. The method of claim 1, further comprising:
identifying at least one alarm from the medical device;
using the determined operational status of the component of the medical device to analyze the identified at least one alarm; and
determining the maintenance procedure for the medical device from the determined operational status and the identified at least one alarm.

4. The method of claim 1, further comprising analyzing the streaming time series medical device data with a plurality of alarm rules to detect alarm events in the streaming time series medical device data.

5. The method of claim 4, wherein the plurality of alarm rules are also applied locally at each of the plurality of monitored medical devices to produce a local alarm at the monitored medical devices.

6. The method of claim 1, further comprising analyzing the streaming time series medical device data to detect procedural events in the time series medical device data.

7. The method of claim 6, further comprising:
determining a prediction of a time at which a current procedure will end; and
scheduling maintenance based upon the prediction.

8. The method of claim 6, further comprising:
aggregating the detected procedural events for each of the plurality of medical devices;
calculating a replacement dates for components of the plurality of medical devices from the aggregated procedural events; and
wherein producing a notification of the determined maintenance procedure comprises scheduling the replacement dates.

9. The method of claim 1, wherein analyzing the streaming time series medical device data further comprises:
monitoring the performance of the component over time;
analyzing a trend in the performance of the component over time; and
predicting a maintenance procedure for the component based upon the trend in performance.

10. The method of claim 9, wherein the component is a battery and the trend in performance is a rate of discharge of the battery when the monitored medical device is on battery power.

11. The method of claim 9, wherein the component is a CO2 absorber, the trend in performance is a measured fraction of expired CO2, and wherein an increase in the measured fraction of expired CO2 is used to predict maintenance of the CO2 absorber.

12. The method of claim 1, wherein producing the notification of the determined maintenance procedure further comprises visually presenting the notification in a graphical user interface on a graphical display.

13. The method of claim 1, further comprising:
storing the streaming time series medical device data in a data lake comprising at least one computer memory;
conducting streaming analytics to apply a plurality of case identification rules to the streaming time series medical device data to identify clinical cases in the streaming time series medical device data;
upon identification of a clinical case in the streaming time series medical device data, storing the identified clinical case in a computer memory; and
conducting streaming analytics to apply a plurality of event detection rules to the streaming time series medical device data to identify events in the streaming time series medical device data.

14. The method of claim 13, further comprising conducting streaming analytics to apply a plurality of operational status rules to the streaming time series medical device data to determine the operational status of the components of the monitored medical devices.

15. The method of claim 14, further comprising applying a plurality of maintenance prediction rules to the determined operational status of the components of the monitored medical devices to determine the maintenance procedure.

16. The method of claim 15, further comprising:
deriving at least one of the plurality of operational status rules by computer learning from the medical device data stored in the data lake; and
deriving at least one of the plurality of maintenance prediction rules by computer learning from the medical device data stored in the data lake.

17. A system for managing maintenance of a medical device by processing streaming time series of medical device data comprises:
data ingestion module that receives streaming time series medical device data and preprocesses the streaming time series medical device data;
a medical device data processor that receives the streaming time series medical device data and applies a plurality of streaming analytics rules to the streaming time series medical device data to identify an operational status of a component of a medical device in the streaming time series medical device data and determine a maintenance procedure for the medical device from the operational status of the component of the medical device; and
an maintenance scheduler that incorporates a schedule of use of the monitored medical devices with a scheduled time the determined maintenance procedure.

18. The system of claim 17, further comprising at least one computer memory comprising a data lake, wherein the received streaming time series medical device data is stored in the at least one computer memory.

19. The system of claim 17 wherein the operational status comprises at least one of component performance monitoring, technical alarm detection, and procedural event detection.

20. The system of claim 19, wherein the determined operational status comprises a component performance, a technical alarm, and procedural event detection and the medical device data processor determines the maintenance procedure from the identified component performance, technical alarms, and procedural event detection.
